(19) United States
(12) Patent Application Publication
(10) Pub. No.: US 2009/0192469 A1
(43) Pub. Date: Jul. 30, 2009

(54) Devices and Methods for Development of a Scar Tissue Tunnel Track

(76) Inventor: Istvan Bognar, Greenville, SC (US)

Correspondence Address: DORITY & MANNING, P.A., POST OFFICE BOX 1449, GREENVILLE, SC, 29602-1449, US

(21) Appl. No.: 12/358,542

(22) Filed: Jan. 23, 2009

Related U.S. Application Data
(60) Provisional application No. 61/023,164, filed on Jan. 24, 2008.

(51) Int. Cl. A61M 5/32

(52) U.S. Cl. 604/177

(57) ABSTRACT

In accordance with one embodiment of the present disclosure, a device for assisting in development of a scar tissue tunnel track is described. The device comprises a base, a strap secured to the base, and a slide pivotally connected to the base. The base is configured to rest on a patient with the strap configured to extend around a portion of the patient to restrict movement of the base. The slide is configured to accommodate a needle and repeatably pivot to a position whereby the needle can be introduced in the patient at a repeatable angle.

### CROSS REFERENCE TO RELATED APPLICATION

The present application is based on and claims priority to U.S. Provisional Application Ser. No. 61/023,164 having a filing date of Jan. 24, 2008, which is incorporated by reference herein.

### BACKGROUND

Constant site cannulation, also know as the buttonhole technique, is a method of cannulating a fistula whereby hemodialysis needles are inserted into the same location at the same angle and depth for each hemodialysis treatment. After cannulating the same site multiple times (approximately 8-10 cannulations in a good wound healer, approximately 12-14 cannulations in a poor or diabetic wound healer) a scar tissue tunnel track will develop at the site. The track is created by formation of scar tissue, analogous to the hole created for a pierced ear, and goes from the surface of the skin to the outside wall of the fistula. Once the track is well healed, it will cause less to no pain and bleeding during subsequent cannulations. After a track is created, a blunt dialysis needle is utilized to decrease the risk of cuts and bleeding to the track.

The technique can be used for new fistulas and also for older accesses and offers many advantages over existing access methods. For instance, constant site cannulation can eliminate the need for anesthetics and allow patients to self-cannulate, thus promoting independence while also reducing complications typically associated with cannulation and prolonging the life of the fistula.

Despite the advantages over other methods of cannulation, constant site cannulation still faces several barriers that have prevented the method from gaining widespread acceptance. One such barrier is the staffing pattern of dialysis clinics. While constant site cannulation is a recommended cannulation method, the technique requires that the same person perform the cannulation on the patient each time until the scar track is formed, which can take as long as 3 to 4 weeks. The staffing pattern of dialysis clinics usually does not allow the same staff member to work with the same patient for the duration needed to create a scar tissue track. Cannulation is a very individualized process as each clinician chooses a unique angle of insertion for a dialysis needle based on independent assessment of the depth of an access. Thus, two different individuals may determine the angle of entry to be slightly different. This can prevent formation of a scar tissue track tunnel and instead lead to damage and bleeding. As a result, one has to recreate a new site if a good scar tissue track tunnel is to be formed.

Thus, a need exists for a device that simplifies the constant site technique for development of a scar tissue tunnel track in a fistula. Methods relating to such a device would also be desirable.

### SUMMARY

In accordance with one embodiment of the present disclosure, a device for assisting in development of a scar tissue tunnel track is described. The device comprises a base, a strap secured to the base, and a slide pivotally connected to the base. The base is configured to rest on a patient with the strap configured to extend around a portion of the patient to restrict movement of the base. The slide is configured to accommodate a needle and repeatably pivot to a position whereby the needle can be introduced in the patient at a repeatable angle.

In another embodiment of the present disclosure, a kit for assisting in development of a scar tissue tunnel track is described. The kit includes a device and a needle. The device comprises a base, a strap secured to the base, and a slide pivotally connected to the base. The base is configured to rest on a patient with the strap configured to extend around a portion of the patient to restrict movement of the base. The slide is configured to accommodate a needle and repeatably pivot to a position whereby the needle can be introduced in the patient at a repeatable angle. The needle is configured to move forward and backward along the slide of the device.

In yet another embodiment of the present disclosure, a method for development of a scar tissue tunnel track is described. The method includes positioning a device on a patient, the device comprising a base, a strap secured to the base, and a slide pivotally connected to the base. The base rests on the patient with the strap extending around a portion of the patient to restrict movement of the base. The slide is pivoted to a repeatable position that can accommodate a needle whereby a needle can be introduced in the patient at a repeatable angle.

Other features and aspects of the present disclosure are discussed in greater detail below.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figures in which:

FIG. 1 illustrates a device in accordance with certain embodiments of the present disclosure; and

FIGS. 2A-2C illustrate a device pivoting in accordance with certain embodiments of the present disclosure.

Repeat use of reference characters in the present specification and drawings is intended to represent same or analogous features or elements of the disclosure.

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

The present disclosure is generally directed to devices and methods that can assist in developing a scar tissue tunnel track. For instance, in certain embodiments of the present disclosure, a device is provided that allows a user to set and consistently reproduce the location of insertion of a needle along a fistula, including the angle of insertion and the distance above the fistula. The present disclosure can eliminate the need for single technician to be present during the period of time required for formation of the scar tissue track.

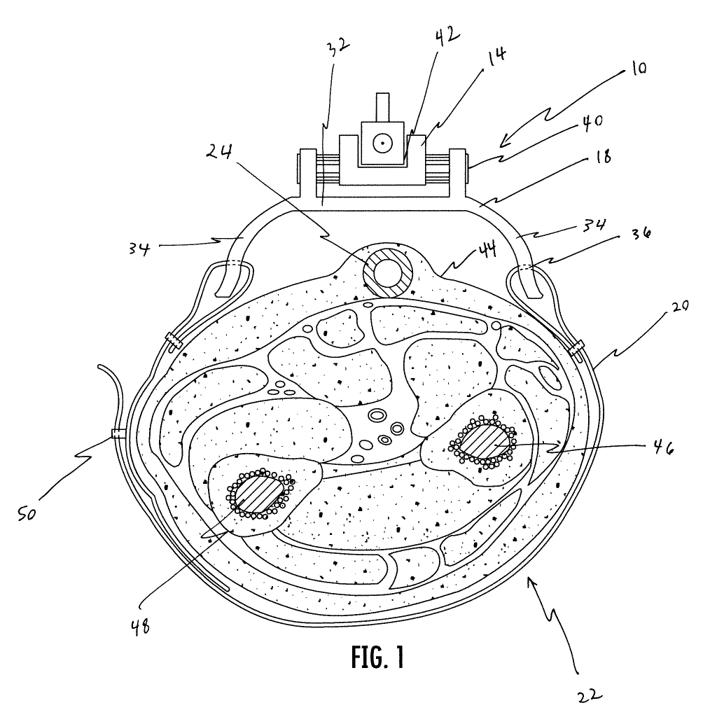

Turning to FIG. 1, a device 10 in accordance with the present disclosure is illustrated. The device 10 includes base 18, strap 20, and slide 14. The base 18 is generally designed to rest on a patient. In this regard, FIG. 1 illustrates base 18 resting on the arm 22 of a patient above fistula 24. From the perspective illustrated in FIG. 1, the device 10 is positioned above fistula 24, which is located under the skin 44 of arm 22 but above the radius 46 and ulna 48 bones. However, it should be understood that device 10 is designed to assist in development of a scar tissue tunnel track at any suitable location of a patient so base 18 is not limited to resting on an arm.

In certain embodiments of the present disclosure, base 18 has a central element 32 and legs 34 that extend from central element 32. The central element 32 and legs 34 can be attached together by methods known in the art or can be integrally formed. Legs 34 can raise central element 32 above fistula 24 to avoid base 18 compressing fistula 24. Legs 34 can define openings 36 for securing strap 20. In certain embodiments, legs 34 can be extended or shortened by a user to increase or decrease the height of central element 32 over fistula 24.

Base 18 can be any suitable size or shape to permit base 18 to rest on a patient above the desired location of a scar tissue tunnel track. For instance, in certain embodiments of the present disclosure, base 18 can be from about 3 cm to about 10 cm in length and from about 2 cm to about 10 cm in width. Base 18 can be formed from any suitable material. For instance, in certain embodiments of the present disclosure, base 18 can be formed from plastic, elastomeric materials, metals, or combinations thereof. In addition, base 18 can be biocompatible.

Strap 20 is secured to base 18 and designed to extend around a portion of a patient (such as arm 22 illustrated in FIG. 1) to limit movement of base 18 on the patient. In certain embodiments of the present disclosure, strap 20 is secured to base 18 through openings 36 in legs 34. However, strap 20 can be secured to base 18 at any suitable location by any suitable method. For instance, strap 20 can be attached to base 18 with adhesive, stitching, or the like or can be molded into base 18. In addition, strap 20 can removably secured to base 18. In this manner, different sized straps can be used in combination with base 18. In addition, strap 20 can be washed separately from base 20, if necessary.

Strap 20 can be formed from two or more separate portions or can be a single element. If strap 20 is made up of more than one portion, each portion can be independently secured to base 18 and removably secured to one another. Strap 20 can also include any suitable fastening mechanism 50 as would be known in the art that can be utilized to tighten strap 20 around a portion of the body of the patient. For instance, suitable fastening mechanisms include hook and loop connectors (such as VELCRO, which is manufactured by Velcro Industries B.V.), button connectors, latch fasteners, magnetic fasteners, or any other suitable fastening mechanism as would be known in the art.

Strap 20 can be any suitable size or shape to secure base 18 to a patient above the desired location of a scar tissue tunnel track. For instance, in certain embodiments of the present disclosure, strap 20 can be from about 5 cm to about 20 cm in length and from about 0.5 cm to about 5 cm in width. Strap 20 can be formed from any suitable material. For instance, in certain embodiments of the present disclosure, strap 20 can be formed from an inflexible plastic, elastomeric materials, fabric, or combinations thereof. In addition, strap 20 can be biocompatible.

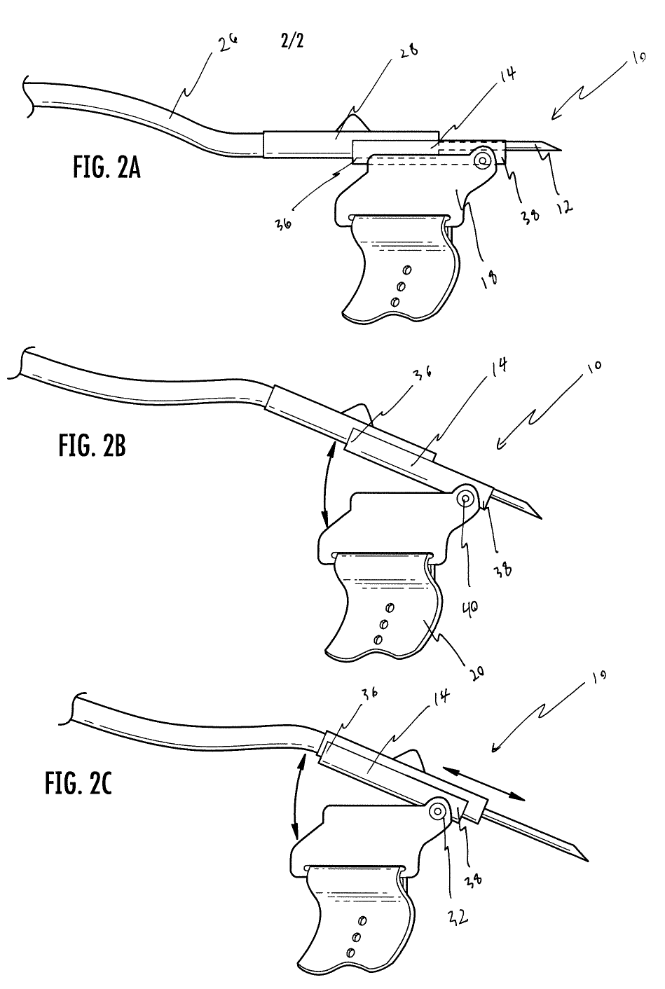

Turning to FIGS. 2A-2C, slide 14 is connected to base 18 and can pivot so that rear portion 36 is angled above forward portion 38. Slide 14 is designed to accommodate a needle 12 whereby the needle 12 can be inserted at a predetermined angle into a patient based on the angle of slide 14. In this manner, the slide 14 can permit consistent entry of the needle 12 in the patient.

Referring again to FIG. 1, slide 14 can be connected to base 18 by any suitable method that permits slide 14 to pivot to a position that is capable of being repeated. For instance, slide 14 can be connected to central portion 32 of base 18 by a graded hinge 40 which can hold slide 14 in place at a desired angle. The grades on the hinge 40 can be designed to make a sound, such as a clicking sound or the like, if desired, based on the angle of pivot of slide 14. However, it should be understood that any suitable method of pivotally connecting slide 14 to base 18 can be utilized, such as a ratchet and pawl type system or the like.

Slide 14 can be any suitable size or shape to accommodate a needle 12 being inserted into a desired location of a scar tissue tunnel track. Slide 14 can define a groove 42 that is sized to allow a needle 12 to move forward from rear portion 36 toward forward portion 38 or backward in the opposite direction but that prevents or limits side to side movement of needle 12. In certain embodiments of the present disclosure, slide 14 can pivot from an angle of about 0 degrees to about 90 degrees and can be from about 3 cm to about 10 cm in length and from about 2 cm to about 10 cm in width. Slide 14 can be formed from any suitable material. For instance, in certain embodiments of the present disclosure, slide 14 can be formed from plastic, elastomeric materials, metals, or combinations thereof. In addition, slide 14 can be biocompatible.

In certain embodiments, the device 10 also includes a needle 12. Any suitable needle 12 can be utilized in connection with the present disclosure, such as a standard hemodialysis needle set. The needle 12 can be moved along slide 14 by way of grip 28 that is located adjacent to needle 12 so as to be above slide 14 when needle 12 is positioned on slide 14. Besides standard needles, in certain embodiments, a needle designed specifically for the device can be utilized. In addition, in certain embodiments, a device and needle can be provided together as a kit.

Referring back to FIGS. 2A-2C, a method for development of a scar tissue tunnel track is illustrated using a device 10 in accordance with the present disclosure. The method can be completed by any trained user of the device, such as a dialysis technician, or even self-administered by a patient. The method includes positioning the device 10 at a suitable location on a patient adjacent to the desired location of the scar tissue tunnel track. If desired, a marking or measurement can be made and recorded as to where the device 10 is being positioned on the patient. As described previously, the base 18 rests on the patient while the strap 20 is extended around a portion of the patient to restrict movement of the base. In this regard, the strap 20 and/or base 18 can include indicia or markings which indicate the level of pressure exerted by the strap 20 and/or the height of the central portion 32 of the base 18 above the surface of the patient so that the same level(s) can be recorded and repeated.

Referring to FIG. 2B, the slide 14 can be pivoted to a suitable angle, either with or without the needle 12 present on the slide 14. Again, the slide 14 and/or base 18 can include indicia or markings which indicate the angle of pivot of the slide so that the same angle can be recorded and repeated. Further, the slide 14 can also include a audible cue which allows a user to record and repeat the position, such as a clicking sound or the like.

Turning to FIG. 2C, the needle 12 is moved forward along the slide 14 and inserted into a patient. Blood is drawn from the patient and travels through tubing 26. After the treatment is complete, the needle 12 and device 10 are removed from the patient. During subsequent cannulations, the device is positioned at the same location on the patient and the slide is moved to the same angle as used on the previous cannulation(s). The needle is once again inserted into the patient at the same location and same angle and can allow for development of a scar tissue tunnel track. In this manner, a scar tissue track can be created with minimal chance of injury or bleeding.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure.

What is claimed is:

1. A device for assisting in development of a scar tissue tunnel track, the device comprising:
a base;
a strap secured to the base; and
a slide pivotally connected to the base;
wherein the base is configured to rest on a patient with the strap configured to extend around a portion of the patient to restrict movement of the base, the slide configured to accommodate a needle and repeatably pivot to a position whereby the needle can be introduced in the patient at a repeatable angle.

2. The device of claim 1, further comprising a needle, the needle configured to move forward and backward along the slide.

3. The device of claim 1, wherein the slide comprises a groove configured to accommodate a needle.

4. The device of claim 1, wherein the strap comprises a fastening mechanism.

5. The device of claim 4, wherein the fastening mechanism comprises hook and loop fasteners.

6. The device of claim 1, wherein the strap is removably secured to the base.

7. The device of claim 1, wherein the strap comprises two separate portions, each portion being independently secured to the base and removably connected to one another.

8. The device of claim 1, wherein the base includes markings to indicate the angle of pivot of the slide.

9. The device of claim 1, wherein the slide includes markings to indicate the angle of pivot of the slide.

10. The device of claim 1, wherein the slide or body is configured to make a sound when the slide pivots a pre-determined distance.

11. A kit for assisting in development of a scar tissue tunnel track, the kit comprising:
a device comprising a base, a strap secured to the base, and
a slide pivotally connected to the base, wherein the base is configured to rest on a patient with the strap configured to extend around a portion of the patient to restrict movement of the base, the slide configured to accommodate a needle and repeatably pivot to a position whereby the needle can be introduced in the patient at a repeatable angle; and
a needle, the needle configured to move forward and backward along the slide of the device.

12. The kit of claim 11, wherein the slide comprises a groove configured to accommodate the needle.

13. The kit of claim 12, wherein the shape of the needle corresponds to the shape of the groove.

14. The kit of claim 11, wherein the strap comprises a fastening mechanism.

15. The kit of claim 11, wherein the strap comprises two separate portions, each portion being independently secured to the base and removably connected to one another.

16. The kit of claim 11, wherein the base includes markings to indicate the angle of pivot of the slide.

17. The kit of claim 11, wherein the slide or body is configured to make a sound when the slide pivots a pre-determined distance.

18. A method for development of a scar tissue tunnel track, the method comprising:
positioning a device on a patient, the device comprising a base, a strap secured to the base, and a slide pivotally connected to the base, wherein the base rests on the patient with the strap extending around a portion of the patient to restrict movement of the base;
pivoting the slide to a repeatable position that can accommodate a needle whereby a needle can be introduced in the patient at a repeatable angle.

19. The method of claim 18, further comprising:
moving a needle forward along the slide at the repeatable position and inserting the needle in the patient at the repeatable angle; and
removing the device and needle from the patient.

20. The method of claim 19, further comprising:
positioning the device on the patient; and
pivoting the slide to the repeatable position and introducing the needle in the patient at the repeatable angle.
